(19) United States
(12) Patent Application Publication
(10) Pub. No.: US 2025/0155364 A1
(43) Pub. Date: May 15, 2025

(54) OPTICAL ANALYSIS ON DIGITAL MICROFLUIDIC (DMF) CARTRIDGES

(71) Applicant: Nicoya Lifesciences Inc., Kitchener (CA)

(72) Inventors: Tanya NARAHARI, Kitchener (CA); Gordon HALL, Cambridge (CA); Krishna IYER, Waterloo (CA); Champika SAMARASEKERA, Guelph (CA); Arjun SUDARSAN, Carlsbad, CA (US)

(21) Appl. No.: 18/837,824

(22) PCT Filed: Mar. 22, 2023
(86) PCT No.: PCT/CA2023/050379

§ 371 Date: Aug. 12, 2024

Related U.S. Application Data
(60) Provisional application No. 63/322,996, filed on Mar. 23, 2022.

(51) Int. Cl. G01N 21/31; G01N 21/05

(52) U.S. Cl. CPC G01N 21/31; G01N 21/05

(57) ABSTRACT

Provided herein are digital microfluidic devices, methods and systems for improving absorbance and/or transmission detection in electromagnetic radiation spectroscopy. For example, devices and methods are provided for determining the absorbance and/or transmission of light when analyzing a fluid (e.g., a droplet) including a target analyte of interest.

### CROSS-REFERENCE

This application claims the benefit of U.S. Provisional Application No. 63/322,996, filed Mar. 23, 2022, which is hereby incorporated by reference in its entirety herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### FIELD

The present invention is directed to digital microfluidic devices, methods and systems for improving absorbance and/or transmission detection in electromagnetic radiation spectroscopy. For example, devices and methods are provided for determining the absorbance and/or transmission of light when analyzing a fluid (e.g., a droplet) including a target analyte of interest.

### BACKGROUND

In digital microfluidics (DMF), it is possible to measure reflectance, fluorescence, chemiluminescence and/or evanescent-waves, for example, using UV, visible, IR or terahertz electromagnetic radiation for interrogation of an analyte-ligand interaction in a sample. For example, it is relatively common to measure reflected or scattered light to quantify interactions between a ligand and an analyte, driven mainly by the fact that only one of the two components of the cartridge is transparent to a range of light wavelengths (typically, the top plate). For instance, a light source and a detector may both be placed on the same side, i.e., over the cartridge (assuming the top plate is transparent to the wavelengths of interest), allowing relatively sensitive, ‘top-read’ measurements to be made. However, many assay types, including, for example, cell assays on DMF, enzyme linked immunosorbent assays (ELISAs) with reporter-substrate reactions (which are quantified via absorbance or optical density), as well as common modes of Fourier-Transform Infrared (FTIR) Spectroscopy, require measurements of transmitted light (i.e., where the light source and detector are on two different sides of the cartridge). Additionally, most DMF systems rely on built-in optics for detection, which drives up the footprint, cost, and complexity of the control hardware. There is a need in the art for DMF cartridges, methods and systems that can leverage off-the-shelf plate readers (e.g., UV-Vis and FTIR spectrophotometers) for analysis of an analyte in solution. Moreover, combining DMF-liquid handling with plate reader analysis will allow universal adaptation of DMF technology, and utilization of DMF detection methods without significant changes to DMF hardware.

In another example, FTIR (Fourier Transform Infrared Spectroscopy) is a technique that can be used to obtain an IR spectrum for a substance using, for example, Michelson interferometry, wherein phase differences (and interference patterns) between waves passing through a sample are measured. While UV/visible-wavelength-spectrometry is useful to identify and quantify molecules in general (and is widely used to read specific colorimetric, chemiluminescence, or fluorometric enzyme-substrate reactions used in ELISAs), the IR spectrum is used to determine molecular structure. Different modes of FTIR include: i) the classic mode, which uses simple transmittance measurements, as well as evanescent-wave modes such as the: ii) Attenuated Total Reflectance (ATR), and iii) spectral reflectance modes. Water produces a high non-specific IR absorbance spectrum, and therefore the classic technique is not very useful for aqueous samples (essentially the preferred matrix for nearly all bio-molecular interactions). There is a need in the art for improved devices, systems and methods for determining molecular structure and/or bio-molecular interactions in aqueous samples. To meet this need, the present invention provides for DMF cartridges, devices, methods and systems that can use evanescent wave techniques to interrogate aqueous samples (or aqueous droplets in DMF devices).

### SUMMARY

In one aspect, the present invention is directed to a digital microfluidic (DMF) cartridge, the cartridge comprising: (a) a bottom plate, the bottom plate comprising a bottom plate substrate and a plurality of electrodes operable to perform droplet operations (e.g., droplet manipulation); (b) a top plate, the top plate comprising a top plate substrate; (c) wherein the top plate and the bottom plate are separated to form a gap; and (d) wherein the bottom plate substrate and/or the top plate substrate comprise a material that is transparent to one or more wavelengths of electromagnetic radiation or wherein the bottom plate substrate and/or top plate substrate comprise a through hole (or window) that is transparent to one or more wavelengths of electromagnetic radiation. In one embodiment, the bottom plate substrate and top plate substrate are made of materials that are transparent to one or more wavelengths of electromagnetic radiation, and the bottom plate, the gap and the top plate comprise a transparent pathway through which one or more wavelengths of electromagnetic radiation can pass.

In some embodiments, the bottom plate substrate is made from a material that is transparent to one or more wavelengths of electromagnetic radiation, wherein the one or more wavelengths of electromagnetic radiation can be selected from x-ray, ultraviolet light, visible light, infrared light, microwave and any combination thereof. In other embodiments, the top plate substrate is made from a material that is transparent to one or more wavelengths of electromagnetic radiation, wherein the one or more wavelengths of electromagnetic radiation can be selected from x-ray, ultraviolet light, visible light, infrared light, microwave and any combination thereof. In still other embodiments, the top plate substrate and the bottom plate substrate are both transparent to one or more wavelengths of electromagnetic radiation selected from x-ray, ultraviolet light, visible light, infrared light, microwave and any combination thereof.

In some embodiments, the material that is transparent to one or more wavelengths is selected from quartz, cyclo olefin polymer (COP), Cyclic olefin copolymer (COC), a ceramic, a multi-layer flexible PCB transmissible to visible light and any combination thereof. In one embodiment, the bottom plate substrate is coated with a transparent conductive material. In another embodiment, the top plate substrate is coated with a transparent conductive material. For example, the transparent conductive material can be indium tin oxide (ITO).

In one embodiment, the bottom plate substrate further comprises a material capable of filtering out one or more wavelengths of electromagnetic radiation (i.e., opaque to one or more wavelengths of electromagnetic radiation). In another embodiment, the top plate substrate further comprises a material capable of filtering out one or more wavelengths of electromagnetic radiation (i.e., opaque to one or more wavelengths of electromagnetic radiation).

In some embodiments, the plurality of electrodes comprises an actuation grid.

In some embodiments, the DMF cartridge has the same dimensions as a standard well plate. In some embodiments, the transparency of the bottom plate substrate and/or top plate substrate coincides with the wells of a standard well plate.

In another aspect, the present invention is directed to a method for analyzing an analyte of interest in a droplet using electromagnetic spectroscopy, the method comprising: (a) providing a DMF cartridge, the DMF cartridge comprising: (i) a bottom plate and a top plate, wherein the bottom plate and the top plate are separated to form a gap; and (ii) a droplet positioned in the gap, wherein the droplet includes an analyte of interest; (b) providing an electromagnetic radiation light source arranged to transmit electromagnetic radiation through the bottom plate, through the droplet, and through the top plate; (c) providing a sensor operable to detect electromagnetic radiation transmitted through the droplet and/or emitted by the droplet; (d) directing electromagnetic radiation from the electromagnetic radiation light source through the bottom plate, through the droplet, through the top plate and to the sensor; (e) detecting the electromagnetic radiation at the sensor; and (f) using a processor, analyzing the analyte of interest in the droplet. In some embodiments, the cartridge useful for the practice of this embodiment can comprise any of the cartridge embodiments disclosed herein.

In some embodiments, the electromagnetic spectroscopy is selected from ultraviolet-visible (UV-Vis) spectroscopy, Fourier-Transform Infrared (FTIR) spectroscopy, Raman spectroscopy, Circular Dichroism (CD) spectroscopy, Near-InfraRed (NIR) spectroscopy, Microfluidic Modulation spectroscopy (MMS) and terahertz spectroscopy.

In some embodiments, the electromagnetic radiation light source is an optical fiber. In some embodiments, the optical fiber is integrated with the DMF cartridge. In some embodiments, the sensor is a spectrophotometer. In some embodiments, the spectrophotometer is integrated with the DMF cartridge.

In yet another aspect, the present invention is directed to a system for analyzing an analyte in a droplet, the system comprising: (a) a DMF cartridge, the DMF cartridge comprising a bottom plate and a top plate, wherein the bottom plate and the top plate are separated to form a gap, and wherein the top plate and/or the bottom plate are made of a material that is transparent to one or more wavelengths of electromagnetic radiation; and (b) an electromagnetic radiation light source capable of emitting electromagnetic radiation, and (c) a sensor capable of detecting electromagnetic radiation. In some embodiments, the cartridge useful for the practice of this embodiment can comprise any of the cartridge embodiments disclosed herein.

In one embodiment, the electromagnetic radiation light source is from a system, wherein the system is an electromagnetic spectroscopy system, and wherein the electromagnetic spectroscopy is selected from ultraviolet-visible (UV-Vis) spectroscopy, Fourier-Transform Infrared (FTIR) spectroscopy, Raman spectroscopy, Circular Dichroism (CD) spectroscopy, Near-InfraRed (NIR) spectroscopy, Microfluidic Modulation spectroscopy (MMS) and terahertz spectroscopy.

In still another aspect, the present invention is directed to a digital microfluidic (DMF) cartridge, the cartridge comprising: (a) a bottom plate, the bottom plate comprising a bottom plate substrate and a plurality of electrodes operable to perform droplet operations (e.g., droplet manipulation); (b) a top plate, the top plate comprising a top plate substrate; and (c) wherein the top plate and the bottom plate are separated by a gap, and wherein the DMF cartridge further comprises an internal reflectance element (IRE) material.

In some embodiments, the internal reflectance element is embedded in the top plate substrate. In other embodiments, the internal reflectance element is embedded in the bottom plate substrate. In still other embodiments, the top plate and/or the bottom plate are made from an internal reflectance element (IRE) material. In yet other embodiments, the internal reflectance element is disposed in the gap between the top plate and the bottom plate.

In some embodiments, the internal reflective element comprises a high-refractive index material, and wherein the high-refractive index material comprises Germanium (Ge), Zinc selenide (ZnSe), silicon, amorphous material transmitting infrared radiation (AMTIR) material, diamond or any combination thereof. In some embodiments, the high-refractive index material comprises a Germanium (Ge) crystal, a Zinc selenide (ZnSe) crystal, a silicon crystal, an amorphous material transmitting infrared radiation (AMTIR) crystal, a diamond crystal or any combination thereof.

In one embodiment, the internal reflectance element (IRE) material is capable of adsorbing an analyte of interest. In another embodiment, the IRE material comprises a surface, and wherein the surface is functionalized with a receptor capable of binding the analyte of interest.

In one embodiment, the receptor is directly bound to the IRE surface, the receptor is directly bound to the crystal surface through hydrogen bonds, hydrophobic interactions or through van der Waals interactions, or the receptor is directly bound to a coating applied to the surface. In still another embodiment, the IRE surface is functionalized by binding the receptor to the IRE surface using one or more linkers, and wherein the one or more linkers include common functional groups used for protein-peptide or aptamer immobilization, including one or more amine (NH2) groups, sulfhydryl (SH) groups, aldehyde (CHO) groups, carboxylate (COO—) groups, or a hydroxyl (OH) groups, or any combination thereof. In one embodiment, the receptor is bound to the IRE surface using cross-linking chemistries such as a NHS ester linkage, a maleimide linkage, a hydrazide linkage, EDC coupling, or a biotin-streptavidin linkage.

In one embodiment, the IRE material comprises a porous material. In another embodiment, the porous material comprises porous silicon photonic crystals or porous tantalum oxide photonic crystals.

In some embodiments, the DMF cartridge further comprises an optical fiber. In some embodiments, the internal reflectance element (IRE) material is embedded in the optical fiber.

In still another aspect, the present invention is directed to a method for analyzing an analyte of interest in a droplet using evanescent-wave-mediated spectroscopy, the method comprising: (a) providing a DMF cartridge, the DMF cartridge comprising: (i) a bottom plate and a top plate, wherein the bottom plate and the top plate are separated to form a gap; (ii) the top plate and/or the bottom plate comprises an embedded internal reflectance element (IRE) material; and (iii) a sample droplet positioned in the gap, wherein the droplet includes an analyte of interest; (b) providing an electromagnetic radiation light source arranged to transmit electromagnetic radiation through the internal reflectance element (IRE) material embedded in the top plate; (c) providing a sensor operable to detect electromagnetic radiation; (d) directing electromagnetic radiation from the electromagnetic radiation light source through the internal reflectance element (IRE) material, thereby creating an evanescent wave which penetrates the sample droplet; (e) detecting the electromagnetic radiation and/or the evanescent wave at the sensor; and (f) using a processor, analyzing the analyte of interest in the sample droplet. In some embodiments, the cartridge useful for the practice of this embodiment can comprise any of the cartridge embodiments disclosed herein.

In one embodiment, the evanescent-wave-mediated spectroscopy is selected from evanescent-wave-mediated ultraviolet-visible (UV-Vis) spectroscopy, evanescent-wave-mediated Fourier-Transform Infrared (FTIR) spectroscopy, evanescent-wave-mediated Raman spectroscopy, evanescent-wave-mediated Circular Dichroism (CD) spectroscopy, evanescent-wave-mediated Near-InfraRed (NIR) spectroscopy, evanescent-wave-mediated Microfluidic Modulation spectroscopy (MMS) and evanescent-wave-mediated terahertz spectroscopy. In another embodiment, the evanescent-wave-mediated spectroscopy comprises evanescent-wave-mediated Fourier-Transform Infrared (FTIR) spectroscopy.

In one embodiment, the depth at which the evanescent wave penetrates the sample droplet depends on the refractive index of the internal reflectance element (IRE) material. In another embodiment, when the evanescent wave penetrates the sample droplet at a depth of from about 0.5 to about 2 μm.

In still another aspect, the present invention is directed to a system for analyzing an analyte in a droplet, the system comprising: (a) a DMF cartridge, the DMF cartridge comprising a bottom plate and a top plate, wherein the bottom plate and the top plate are separated to form a gap, and wherein the top plate and/or the bottom plate further comprise an embedded internal reflectance element (IRE) material; and (b) an electromagnetic radiation light source capable of emitting electromagnetic radiation into the internal reflectance element (IRE) material, thereby producing an evanescent wave capable of penetrating the droplet; and (c) a sensor capable of detecting the electromagnetic radiation and/or the evanescent wave. In some embodiments, the cartridge useful for the practice of this embodiment can comprise any of the cartridge embodiments disclosed herein.

In one embodiment, the electromagnetic radiation light source is from a system, the system comprising an electromagnetic spectroscopy system, and wherein the electromagnetic spectroscopy is selected from ultraviolet-visible (UV-Vis) spectroscopy, Fourier-Transform Infrared (FTIR) spectroscopy, Raman spectroscopy, Circular Dichroism (CD) spectroscopy, Near-InfraRed (NIR) spectroscopy, Microfluidic Modulation spectroscopy (MMS) and terahertz spectroscopy.

In yet another aspect, the present invention is directed to a method for analyzing an analyte of interest in a sample fluid using evanescent-wave-mediated spectroscopy, the method comprising: (a) providing an optical fiber, the optical fiber comprising: (i) a hollow channel, wherein the hollow channel comprises an internal reflectance element (IRE) material; and (ii) a sample fluid positioned within the hollow channel, wherein the sample fluid includes an analyte of interest; (b) providing an electromagnetic radiation light source arranged to transmit electromagnetic radiation through the internal reflectance element (IRE) material; (c) providing a sensor operable to detect the electromagnetic radiation; (d) directing electromagnetic radiation from the electromagnetic radiation light source through the internal reflectance element (IRE) material, thereby creating an evanescent wave which penetrates the sample fluid; (e) detecting the electromagnetic radiation and/or the evanescent wave at the sensor; and (f) using a processor, analyzing the analyte of interest in the sample fluid.

In one embodiment, the hollow channel is made from an internal reflectance element (IRE) material. In another embodiment, the internal reference element (IRE) material is embedded in the hollow channel. In still another embodiment, the IRE material comprises a porous material with a suitable refractive index. In yet another embodiment, the porous material comprises porous silicon photonic crystals or porous tantalum oxide photonic crystals.

In some embodiments, the internal reflective element comprises a high-refractive index material, and wherein the high-refractive index material comprises Germanium (Ge), Zinc selenide (ZnSe), silicon, amorphous material transmitting infrared radiation (AMTIR) material, diamond or any combination thereof. In some embodiments, the high-refractive index material comprises a Germanium (Ge) crystal, a Zinc selenide (ZnSe) crystal, a silicon crystal, an amorphous material transmitting infrared radiation (AMTIR) crystal, a diamond crystal or any combination thereof.

In one embodiment, the internal reflectance element (IRE) material is capable of adsorbing an analyte of interest. In another embodiment, the IRE material comprises a surface, and wherein the surface is functionalized with a receptor capable of binding the analyte of interest.

In one embodiment, the receptor is directly bound to the IRE surface, the receptor is directly bound to the crystal surface through hydrogen bonds, hydrophobic interactions or through van der Waals interactions, or the receptor is directly bound to a coating applied to the surface. In another embodiment, the sensor is functionalized by binding the receptor to the IRE surface using one or more linkers, and wherein the one or more linkers include common functional groups used for protein-peptide or aptamer immobilization, including one or more amine (NH2) groups, sulfhydryl (SH) groups, aldehyde (CHO) groups, carboxylate (COO—) groups, or a hydroxyl (OH) groups, or any combination thereof. In another embodiment, the receptor is bound to the IRE surface using cross-linking chemistries such as an NHS ester linkage, a maleimide linkage, a hydrazide linkage, EDC coupling, or a biotin-streptavidin linkage.

In one embodiment, the evanescent-wave-mediated spectroscopy is selected from evanescent-wave-mediated ultraviolet-visible (UV-Vis) spectroscopy, evanescent-wave-mediated Fourier-Transform Infrared (FTIR) spectroscopy, evanescent-wave-mediated Raman spectroscopy, evanescent-wave-mediated Circular Dichroism (CD) spectroscopy, evanescent-wave-mediated Near-InfraRed (NIR) spectroscopy, evanescent-wave-mediated Microfluidic Modulation spectroscopy (MMS) and evanescent-wave-mediated terahertz spectroscopy. In another embodiment, the evanescent-wave-mediated spectroscopy comprises evanescent-wave-mediated Fourier-Transform Infrared (FTIR) spectroscopy.

In one embodiment, the depth at which the evanescent wave penetrates the sample droplet depends on the refractive index of the crystal internal reflectance element (IRE) surface. In another embodiment, when the evanescent wave penetrates the sample droplet at a depth of from about 0.5 to about 2 μm.

In accordance with some aspects of the present invention, the method can be used for surface plasmon resonance (SPR) and FTIR.

In still yet another aspect, the present invention is directed to a system for analyzing an analyte in a sample fluid, the system comprising: (a) an optical fiber, the optical fiber comprising a hollow channel wherein the hollow channel comprises an internal reflectance element (IRE) material; and (b) an electromagnetic radiation light source capable of emitting electromagnetic radiation into the internal reflectance element (IRE) material, thereby producing an evanescent wave capable of penetrating the sample fluid; (c) a sensor capable of detecting the electromagnetic radiation and/or the evanescent wave; (d) optionally, a fluidic pump operable to pull fluid through the hollow channel of the optical fiber; and (e) optionally, one or more sample fiber couplers.

In one embodiment, the hollow channel is made from an internal reflectance element (IRE) material. In another embodiment, the internal reference element (IRE) material is embedded in the hollow channel. In still another embodiment, the IRE material comprises a porous material with a suitable refractive index. In yet another embodiment, the porous material comprises porous silicon photonic crystals or porous tantalum oxide photonic crystals.

In some embodiments, the electromagnetic radiation light source is from a system, the system comprising an electromagnetic spectroscopy system, and wherein the electromagnetic spectroscopy is selected from ultraviolet-visible (UV-Vis) spectroscopy, Fourier-Transform Infrared (FTIR) spectroscopy, Raman spectroscopy, Circular Dichroism (CD) spectroscopy, Near-InfraRed (NIR) spectroscopy, Microfluidic Modulation spectroscopy (MMS) and terahertz spectroscopy.

In some embodiments, the internal reflective element comprises a high-refractive index material, and wherein the high-refractive index material comprises Germanium (Ge), Zinc selenide (ZnSe), silicon, amorphous material transmitting infrared radiation (AMTIR) material, diamond or any combination thereof. In some embodiments, the high-refractive index material comprises a Germanium (Ge) crystal, a Zinc selenide (ZnSe) crystal, a silicon crystal, an amorphous material transmitting infrared radiation (AMTIR) crystal, a diamond crystal or any combination thereof.

In one embodiment, the internal reflectance element (IRE) material is capable of adsorbing an analyte of interest. In another embodiment, the IRE material comprises a surface, and wherein the surface is functionalized with a receptor capable of binding the analyte of interest.

In one embodiment, the receptor is directly bound to the IRE surface, the receptor is directly bound to the crystal surface through hydrogen bonds, hydrophobic interactions or through van der Waals interactions, or the receptor is directly bound to a coating applied to the surface. In another embodiment, the sensor is functionalized by binding the receptor to the IRE surface using one or more linkers, and wherein the one or more linkers include common functional groups used for protein-peptide or aptamer immobilization, including one or more amine (NH2) groups, sulfhydryl (SH) groups, aldehyde (CHO) groups, carboxylate (COO—) groups, or a hydroxyl (OH) groups, or any combination thereof. In still another embodiment, the receptor is bound to the IRE surface using cross-linking chemistries such as an NHS ester linkage, a maleimide linkage, a hydrazide linkage, EDC coupling, or a biotin-streptavidin linkage.

In some embodiments, the system has dual surface plasmon resonance (SPR) and FTIR functionality.

In still another aspect, the present invention is directed to a method for analyzing an analyte of interest in a droplet, the method comprising: (a) providing a DMF cartridge, the DMF cartridge comprising: (i) a bottom plate and a top plate, wherein the bottom plate and the top plate are separated to form a gap, and wherein the gap includes an optical element operable to refract light from a light source; and (ii) a droplet positioned in the gap, wherein the droplet includes an analyte of interest; (b) providing a light source and a sensor arranged co-planar with bottom plate and top plate, wherein the light source is operable to transmit light through the droplet and wherein the sensor is operable to detect light from the droplet, and wherein the optical element in the gap elongates the light path through the droplet; (c) directing light from the light source through the droplet and to the sensor; (d) detecting the light at the sensor; and (e) using a processor, analyzing the analyte of interest in the droplet.

In some embodiments, the optical element comprises an optically active material, and wherein the optically active material is deposited or doped onto the top plate. In some embodiments, the optical element comprises a prism, and wherein the prism is operable to refract light from a horizontal light path to a perpendicular light path. In other embodiments, the optical element comprises a prism, and wherein the prism is operable to refract light from a perpendicular light path to a planar light path. In other embodiments, the prism comprises a mirrored prism or a dichroic prism.

In one embodiment, the optical element comprises a beamsplitter operable to split the light in the gap into two light beams.

In one embodiment, the optical element comprises a series of two or more prisms. For example, the series of two or more prisms comprises two or more dichroic prisms with wavelength dependent properties.

In some embodiments, the optical element comprises a reflective curved surface, and wherein the reflective curved surface operates to focus the light to a single point within the gap. In one embodiment, the reflective curved surface further operates to focus the light through an aperture in the bottom plate and onto the sensor.

In some embodiments, the light source is an optical fiber. In some embodiments, the optical fiber is integrated with the DMF cartridge. In some embodiments, the sensor is a spectrophotometer. In some embodiments, the spectrophotometer is integrated with the DMF cartridge.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates an exemplary digital microfluidics (DMF) system for optical analysis of a sample fluid containing a target analyte.

FIGS. 2A-2B illustrates an exemplary absorbance detection device, a DMF cartridge, for optical analysis of a fluid (e.g., droplets) containing a target analyte, in accordance with one aspect of the invention. As shown in FIG. 2A, the illustrated DMF cartridge includes transparent top and bottom plates allowing for multimodal measurements (i.e., obtaining measurements from either the top or bottom of the DMF cartridge). FIG. 2B illustrates a DMF cartridge comprising a top plate and bottom plate having a UV-visible or infrared optical filter.

FIG. 3 illustrates a DMF plate reader workflow in accordance with one embodiment of the present invention. As shown in FIG. 3, in accordance with one embodiment, the workflow utilizes a DMF cartridge that meets the ANSI standards for 96 well plates, including cartridge dimensions, well size and well spacing, and a control unit to control processing. Once a sample is processed by the controller, the DMF cartridge can be transferred to a plate reader for multimode, multi-wavelength, optical analysis of any kind (e.g., UV-Vis, Vis, IR, and terahertz).

FIG. 4 illustrates a method for optical analysis of an analyte of interest in a droplet using electromagnetic spectroscopy, in accordance with one aspect of the invention. As shown in FIG. 4, the method utilizes a DMF cartridge that has been made out of a material that is transparent to one or more wavelengths of electromagnetic radiation.

FIGS. 5A-5B illustrate an exemplary detection device, a DMF cartridge, for optical analysis using evanescent-wave-mediated Fourier-Transform Infrared (FTIR) spectroscopy of a fluid (e.g., droplets) containing a target analyte, in accordance with another embodiment of the invention. As shown in FIGS. 5A-5B, the DMF cartridge includes a top plate comprising an internal reflectance element (IRE) material.

FIG. 6A-6C illustrate an exemplary detection device, a DMF cartridge, for optical analysis using evanescent-wave-mediated Fourier-Transform Infrared (FTIR) spectroscopy of a fluid (e.g., droplets) containing a target analyte, in accordance with another embodiment of the invention. As shown in FIGS. 6A-6C, the DMF cartridge includes a top plate comprising one or more internal reflectance element (IRE) materials.

FIG. 6D illustrates an exemplary detection device, a DMF cartridge, for optical analysis using evanescent-wave-mediated Fourier-Transform Infrared (FTIR) spectroscopy of a fluid (e.g., droplets) containing a target analyte, in accordance with yet another embodiment of the invention. As shown in FIG. 6D, the DMF cartridge includes a top plate having an internal reflectance element (IRE) material comprising a porous material to maximize the surface area available for ligand capture.

FIG. 7 illustrates a method for optical analysis using Fourier-Transform Infrared (FTIR) spectroscopy of an analyte of interest in a droplet, in accordance with one aspect of the invention. As shown in FIG. 7, the method utilizes a DMF cartridge that has a top plate including an embedded internal reflectance element (IRE) surface.

FIGS. 8A-8B illustrate exemplary hollow core optical fiber arrangements useful for the practice of one embodiment of the present invention. As shown in FIGS. 8A-8B, hollow core optical fiber arrangements include a hollow core fiber, a sample droplet, a pump, a light source and a sensor or detector.

FIG. 9A-9C illustrate exemplary operations of the systems of FIGS. 8A-8B, with optional pump, for fluid transport through the hollow core fiber.

FIG. 10 illustrates Microfluidic modulation spectroscopy, a technique where a sample and a reference solvent stream are rapidly modulated through a microfluidic cell in a DMF device, in accordance with one aspect of the present invention.

FIG. 11 illustrates a method for optical analysis using electromagnetic spectroscopy of an analyte of interest in a droplet, in accordance with one aspect of the invention. As shown in FIG. 11, the method utilizes a DMF cartridge comprising a bottom plate, a top plate and a gap between the bottom and top plate, wherein the gap further comprises an optical element operable to refract a light path and thereby elongate the light path through the fluid.

FIG. 12 illustrates an exemplary absorbance detection device, a DMF cartridge, for optical analysis of an analyte of interest in a droplet using electromagnetic spectroscopy, in accordance with one aspect of the invention. As shown in FIG. 12, the exemplary DMF cartridge includes an optical element operable to refract light from a light source.

FIG. 13A-B illustrate exemplary detection devices, including DMF cartridges for optical analysis of an analyte of interest in a droplet using electromagnetic spectroscopy, in accordance with another embodiment of the invention. As shown in FIG. 13A, the exemplary DMF cartridge includes a prism to refract light from a perpendicular (or vertical) light path to a horizontal (or planar) light path. Whereas in FIG. 13B, the exemplary DMF cartridge includes a prism that refracts light from a horizontal (or planar) light path to a perpendicular (or vertical) light path.

FIG. 14 illustrates an exemplary detection device, a DMF cartridge, for optical analysis of an analyte of interest in a droplet using electromagnetic spectroscopy, in accordance with another embodiment of the invention. As shown in FIG. 14, the exemplary DMF cartridge includes a series of prisms to refract light from a light source.

FIG. 15 illustrates an exemplary detection device, a DMF cartridge, for optical analysis of an analyte of interest in a droplet using electromagnetic spectroscopy, in accordance with another embodiment of the invention. As shown in FIG. 15, the exemplary DMF cartridge includes a reflective curved surface operative to focus the light to a single point within the gap.

FIG. 16 illustrates an exemplary detection device, a DMF cartridge, for optical analysis of an analyte of interest in a droplet using electromagnetic spectroscopy, in accordance with another embodiment of the invention. As shown in FIG. 16, the exemplary DMF cartridge includes an aperture operable to focus the light onto the sensor.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Example Digital Microfluidics (DMF) System

In some embodiments, a DMF system useful to practice the present invention may include the DMF cartridge, an optical detection system, and a controller. The optical detection system may include, for example, an illumination source and an optical measurement device in relation to the sensor elements. In some embodiments, the optical detection system may operate in absorbance mode while in other embodiments, the optical detection system may operate in transmission mode. The controller may be provided for controlling fluid manipulation (e.g., droplet manipulation) by activating/deactivating electrodes (or pads) in the DMF cartridge. The controller also may manage the overall operations of the DMF system.

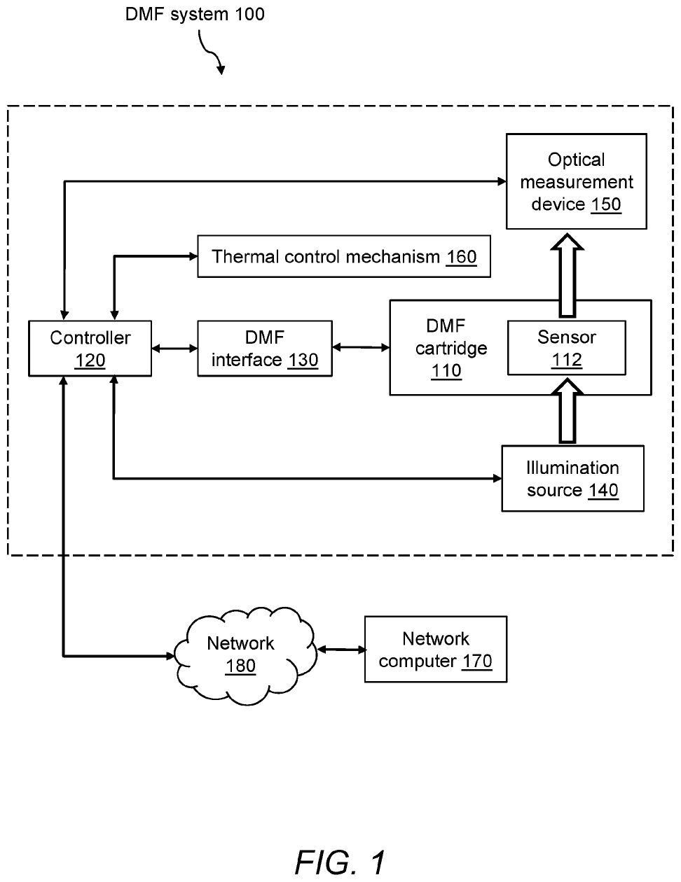

In one embodiment, as shown in FIG. 1, a block diagram of an embodiment of the presently disclosed DMF system 100 that includes a DMF cartridge 110 that may include a sensor 112 for analysis of a target analyte. In DMF system 100 for analysis of a target analyte, analysis can mean, for example, detection, identification, quantification, or measuring analytes and/or the interactions of analytes with other substances, such as binding kinetics. Exemplary analytes may include, but are not limited to, small molecules, proteins, peptides, antibodies, nucleic acids, atoms, ions, polymers, and the like.

DMF cartridge 110 may facilitate DMF capabilities generally for fluidic actuation including droplet merging, splitting, dispensing, diluting, and the like. These DMF capabilities can be used for sample preparation, as is well known in the art. For example, one application of these DMF capabilities may be fluid splitting of a ligand containing fluid allowing a first portion of the ligand containing fluid to be used for ligand immobilization to the sensor and allowing a second portion of the ligand containing fluid to be used in a dissociation phase or dissociation step for determining a dissociation rate constant. In still other embodiments, the DMF capabilities may be used for other processes, such as waste removal. DMF cartridge 110 of DMF system 100 can be provided, for example, as a disposable and/or reusable cartridge. More details and/or capabilities of DMF cartridges are described hereinbelow.

While the discussion presented herein may involve use of a DMF system 100 and DMF sensor 112, it is contemplated that other systems and means for interrogating and analyzing a fluid can also be used in place of or in addition the DMF system disclosed herein. For example, as is well known in the art, and as described elsewhere herein, other known systems for absorbance (i.e., for measurements related to absorbed light) and/or transmission (i.e., for measurements of transmitted light) interrogation of a fluid can be used. In some embodiments, the system used in the practice of the present invention is an electromagnetic radiation spectroscopy system, such as, ultraviolet-visible (UV-Vis) spectroscopy, Fourier-Transform Infrared (FTIR) spectroscopy, Raman spectroscopy, Circular Dichroism (CD) spectroscopy, Near-InfraRed (NIR) spectroscopy, Microfluidic Modulation spectroscopy (MMS) or terahertz spectroscopy. In one embodiment, the system used for practice of the present invention is an off-the-shelf electromagnetic radiation spectroscopy system. In general, the devices, DMF cartridges and methods described herein can leverage off-the-shelf plate readers (e.g., UV-Vis and FTIR spectrophotometers) for analysis of an analyte in a fluid.

The DMF system 100 may further include a controller 120, a DMF interface 130, an illumination source 140, an optical measurement device 150, and optionally a thermal control mechanism 160. Controller 120 may be electrically coupled to the various hardware components of DMF system 100, such as to DMF cartridge 110, illumination source 140, and an optical measurement device 150. In particular, controller 120 may be electrically coupled to DMF cartridge 110 via DMF interface 130, wherein DMF interface 130 may be, for example, a pluggable interface for connecting mechanically and electrically to DMF cartridge 110.

Controller 120 may, for example, be a general-purpose computer, special purpose computer, personal computer, microprocessor, or other programmable data processing apparatus. Controller 120 may provide processing capabilities, such as storing, interpreting, and/or executing software instructions, as well as controlling the overall operations of DMF system 100. The software instructions may comprise machine readable code stored in non-transitory memory that is accessible by the controller 120 for the execution of the instructions. Controller 120 may be configured and programmed to control data and/or power aspects of these devices. For example, with respect to DMF cartridge 110, controller 120 may control fluid operations and/or droplet manipulation by activating/deactivating electrodes. Generally, controller 120 can be used for any functions of the DMF system 100. For example, controller 120 can be used to authenticate the DMF cartridge 110 in a fashion similar to how printer manufacturers check for their branded ink cartridges, controller 120 can be used to verify that the DMF cartridge 110 is not expired, controller 120 can be used to confirm the cleanliness of the DMF cartridge 110 by running a certain protocol for that purpose, and so on.

Additionally, in some embodiments, DMF cartridge 110 may include capacitive feedback sensing. For example, a signal may be generated or detected by a capacitive sensor that can detect droplet position, velocity, and size. Further, in other embodiments, instead of capacitive feedback sensing, DMF cartridge 110 may include a camera or other optical device to provide an optical measurement of the droplet position, velocity, and size, which can trigger controller 120 to re-route the droplets at appropriate positions. The feedback can be used to create a closed-loop control system to optimize droplet actuation rate and verify droplet operations are completed successfully.

Optionally, DMF system 100 can be connected to a network. For example, controller 120 may be in communication with a networked computer 170 via a network 180. Networked computer 170 can be, for example, any centralized server or cloud server. Network 180 can be, for example, a local area network (LAN) or wide area network (WAN) for connecting to the internet.

In DMF system 100, illumination source 140 and optical measurement device 150 may be arranged with respect to the sensor 112 (e.g., fixed PR sensing and/or in-solution PR sensing) of DMF cartridge 110. The illumination source 140 may be, for example, a light source for the visible range (400-800 nm), such as, but not limited to, a white light-emitting diode (LED), a halogen bulb, an arc lamp, an incandescent lamp, lasers, and the like. Illumination source 140 is not limited to a white light source. Illumination source 140 may be any color light that is useful in DMF system 100. Optical measurement device 150 may be used to obtain DMF light intensity readings for determining absorbance and/or transmission of light. Optical measurement device 150 may be, for example, a charge coupled device, a photodetector, a spectrometer, a photodiode array, or any combinations thereof. Further, DMF system 100 is not limited to one illumination source 140 and one optical measurement device 150 only. DMF system 100 may include multiple illumination sources 140 and/or multiple optical measurement devices 150 to support multiple sensors. Optional thermal control mechanisms 160 may be any mechanisms for controlling the operating temperature of DMF cartridge 110.

### Devices, Methods and Systems for Optical Analysis of a Sample Fluid

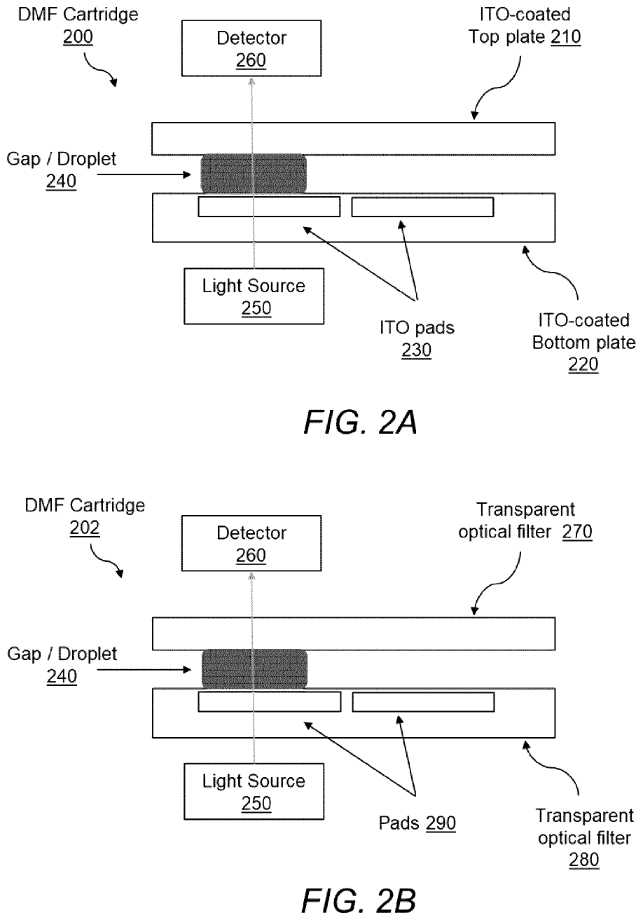

FIGS. 2A-2B illustrates an exemplary absorbance detection device, a DMF cartridge, for optical analysis of a fluid (e.g., droplets) containing a target analyte, in accordance with one aspect of the invention. As shown in FIG. 2A, the illustrated DMF cartridge includes transparent top and bottom plates allowing for multimodal measurements (i.e., obtaining measurements from either the top or bottom of the DMF cartridge). In other embodiments, the top and bottom plates can contain a transparent through hole, or window, that is transparent to one or more wavelengths of electromagnetic radiation. The use of a material that is transparent to one or more wavelengths of electromagnetic radiation in the top and/or bottom plate, or the use of a transparent through hole or window in top and/or bottom plates, allows for electromagnetic radiation at one or more wavelengths to pass through the top and/or bottom plates (or through hole therein) for interrogation of an analyte in a fluid.

FIG. 2B illustrates a DMF cartridge comprising a top plate and bottom plate having a UV-visible or infrared optical filter. In accordance with this embodiment, the UV-visible or infrared optical filter can operate to filter out, or block, UV-visible or infrared electromagnetic radiation, and thereby, allow for interrogation of an analyte with a specific wavelength of electromagnetic radiation.

More specifically, the DMF cartridge 202 of FIG. 2A comprises a transparent cartridge that includes a top plate 210 made using a transparent top plate substrate or material that is coated indium tin oxide (ITO), a bottom plate 220 made using a transparent bottom plate substrate or material that is coated with ITO and a plurality of electrodes (or pads) 230 operable to perform droplet operations (e.g., droplet manipulation). In accordance with the present invention, the use of a DMF cartridge with transparent top and bottom plates provides a transparent pathway for light (e.g., one or more wavelengths of electromagnetic radiation) from a light source 250 to pass through the bottom plate 220, through the gap 240 (including a droplet), through the top plate 210, and subsequently to the sensor or detector 260, thereby allowing one to measure transmitted light or absorbance. In another embodiment, light (e.g., one or more wavelengths of electromagnetic radiation) can pass from a light source through the top plate, through the gap (including a droplet), through the bottom plate, and subsequently to the sensor or detector. In general, any known material that is transparent to one or more wavelengths of electromagnetic radiation can be used as the top plate substrate or the bottom plate substrate. For example, the transparent top plate substrate and/or bottom plates substrate can be selected from a UV/vis/IR transparent material, such as quartz (for UV transparency) or cyclo olefin polymer (COP), Cyclic olefin copolymer (COC) (for UV-visible-wavelength transparency), ceramics, or multi-layer flexible PCBs (for visible-wavelength transparency), or the regular FR4/prepreg for IR transparency. As would be readily understood by one of skill in the art, the transparent material used for the top plate and/or bottom plate can be selected based on the anticipated use (e.g., based on the wavelength desired for interrogation of a given target analyte). The DMF cartridge further includes a gap (containing a droplet therein) 240 that is formed by the separation or spacing between the top plate and the bottom plate and, as noted above, the bottom plate includes a plurality of electrodes for droplet manipulation. In another embodiment, the DMF cartridge can be patterned with an actuation grid for droplet manipulation.

Although the embodiment shown in FIG. 2A includes the use of transparent top and bottom plates (i.e., transparent to one or more wavelengths of electromagnetic radiation), other embodiments are envisioned herein. For example, in one embodiment, the top plate and/or bottom plate are made from a material that is transparent to one or more wavelengths of electromagnetic radiation. In an alternative embodiment, the top and/or bottom plates contain a through hole, or window, therein that allow passage of electromagnetic radiation at one or more wavelengths.

As shown in FIG. 2B, in another embodiment, the DMF cartridge 202 includes a top plate 270 made from a transparent top plate substrate or material, a bottom plate 280 made from a transparent top plate substrate or material and a plurality of electrodes (or pads) 290 operable to perform droplet operations (e.g., droplet manipulation). In accordance with one embodiment of the present invention, as shown in FIG. 2B, the use of a DMF cartridge with transparent top and bottom plates provides a transparent pathway for light (e.g., one or more wavelengths of electromagnetic radiation) from a light source 250 to pass through the transparent bottom plate 280, through the gap 240, through the transparent top plate 270, and subsequently to the sensor or detector 260.

Furthermore, in some embodiments, the DMF cartridge 202 can be made from a material selected to provide an optical filter (i.e., that blocks or partially blocks one or more specific wavelengths of electromagnetic radiation). For example, a material can be selected for either, or both, of the top plate substrate or the bottom plate substrate that confers the ability to filter our incident and/or transmitted/emitted light.

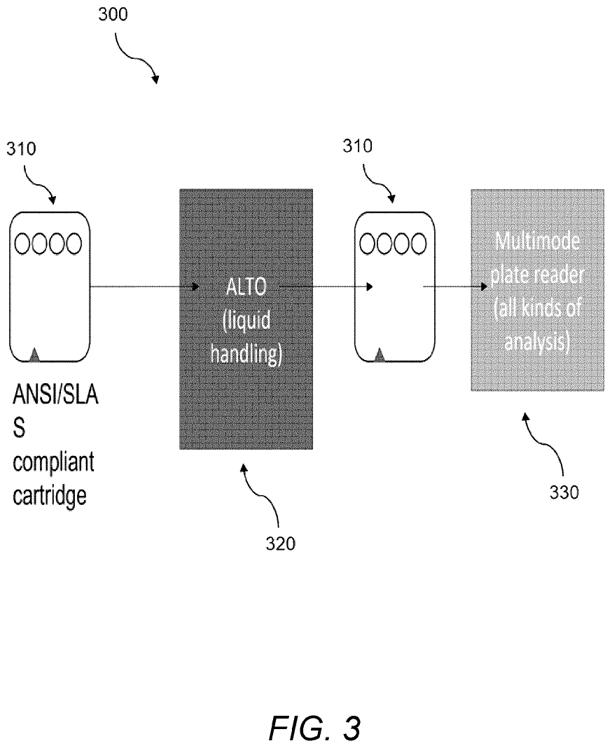

FIG. 3 illustrates a DMF plate reader workflow in accordance with one embodiment of the present invention. As shown in FIG. 3, the method utilizes a DMF cartridge 310 that has been made out of a material that is transparent to one or more wavelengths of electromagnetic radiation, a liquid handling processor or DMF control unit 320, and a multimode plate reader 330 for multimode, multi-wavelength, optical analysis of any kind (e.g., UV, Vis, IR or terahertz spectroscopy) once the sample is processed the DMF cartridge is transferred to the multimode plate reader.

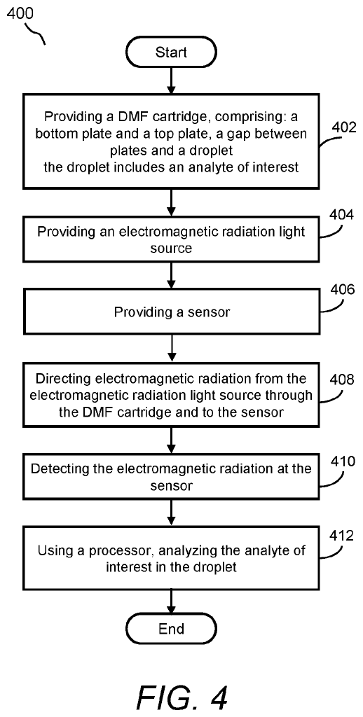

FIG. 4 illustrates a method for optical analysis of an analyte of interest in a droplet using electromagnetic spectroscopy, in accordance with one aspect of the invention. As shown in FIG. 4, the method utilizes a DMF cartridge that has been made out of a material that is transparent to one or more wavelengths of electromagnetic radiation.

At step 402, a DMF cartridge is provided. In accordance with this aspect of the invention, the DMF cartridge comprising: (i) a bottom plate and a top plate, wherein the bottom plate and the top plate are separated to form a gap; and (ii) a droplet positioned in the gap, wherein the droplet includes an analyte of interest. In general, any DMF cartridge disclosed herein can be used in the practice of this method. In particular, any DMF cartridge comprising a transparent top plate and/or a transparent bottom plate can be used, such as those disclosed herein in conjunction with FIGS. 2A and 2B.

At step 404, an electromagnetic radiation light source is provided. The electromagnetic radiation source is arranged to transmit electromagnetic radiation through the bottom plate, through the droplet, and through the top plate. In accordance with this aspect of the invention, any known means for analyzing electromagnetic radiation can be used. For example, electromagnetic spectroscopy can be used, such as ultraviolet-visible (UV-Vis) spectroscopy, Fourier-Transform Infrared (FTIR) spectroscopy, Raman spectroscopy, Circular Dichroism (CD) spectroscopy, Near-InfraRed (NIR) spectroscopy, Microfluidic Modulation spectroscopy (MMS) or terahertz spectroscopy for analysis.

At step 406, a sensor is provided, wherein the sensor is operable to detect electromagnetic radiation transmitted through the droplet and/or emitted by the droplet.

At step 408, electromagnetic radiation is directed from the electromagnetic radiation light source through the bottom plate, through the droplet (e.g., in the gap between the bottom and top plates), subsequently through the top plate and to the sensor. Alternatively, in some embodiments electromagnetic radiation can be directed from the electromagnetic radiation light source through the top plate, through the droplet (e.g., in the gap between the top and bottom plates), subsequently through the bottom plate and to the sensor.

At step 410, electromagnetic radiation is detected at the sensor.

At step 412, a processor is used to analyze an analyte of interest in the droplet.

In another aspect of the present invention, Fourier Transform Infrared Spectroscopy (FTIR) can be used to obtain an IR spectrum for a substance using Michelson interferometry, wherein phase differences (and interference patterns) between waves passing through a sample are measured. While UV/visible-wavelength spectrometry is useful to identify and quantify molecules in general (and is widely used to read specific colorimetric, or fluorometric enzyme-substrate reactions used in ELISAs), the IR spectrum is typically used to determine molecular structure. Different modes of FTIR can include: i) the classic mode, which uses simple transmittance measurements, as well as evanescent-wave modes such as the: ii) Attenuated Total Reflectance (ATR), and iii) spectral reflectance modes. As one of skill in the art is aware, water may produce a high non-specific IR absorbance spectrum, and therefore prior art technique may not be ideal for aqueous samples. The present invention by contrast uses evanescent wave techniques to probe aqueous samples (or aqueous droplets in DMF devices). The general mechanism of the evanescent wave technique is as follows: light travels through a high refractive-index crystal (such as Ge, ZnSe, Silicon, AMTIR, or diamond), also known as the Internal Reflectance Element (IRE). As it undergoes total internal reflection within the IRE, it produces an evanescent wave, which penetrates the sample in contact with the crystal to a depth from about 0.5 μm to about 2 μm, depending on the refractive index of the crystal. Therefore, an analyte which adsorbs to the surface of the IRE (and is therefore present at the interface) may be probed, eliminating any background from the aqueous bulk.

Evanescent-wave FTIR can be performed on DMF using a suitable IRE surface that can either adsorb an analyte-of-interest non-specifically, or be functionalized with an analyte-specific receptor molecule. Further, several modes of functionalization may be used with different IRE-types depending on the depth of wave-penetration into the sample conferred by these IREs. The functionalized crystal might be within the cartridge top or bottom plate, or it might be located within an optical-fiber-sensor (similar to the SPR-fiber sensors patented by Nicoya, but with different composition and functionality). The fiber sensor might also have dual SPR/FTIR functionality.

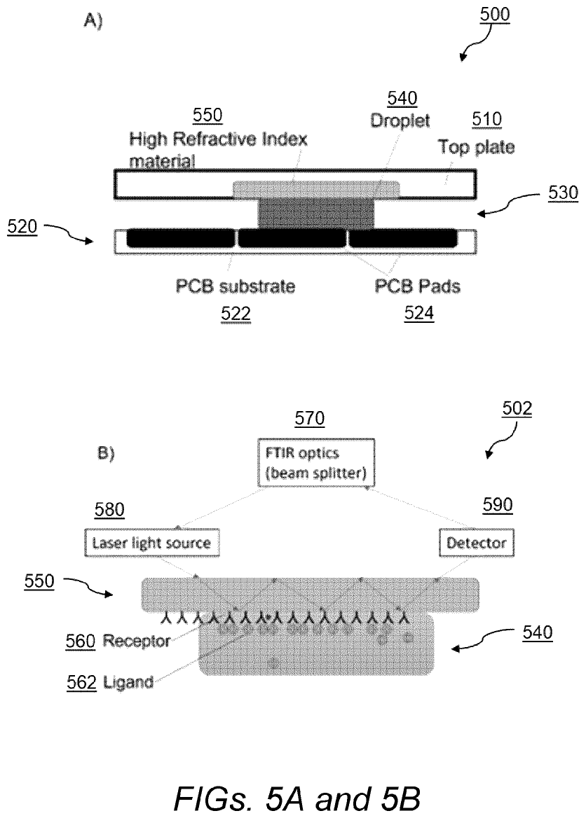

FIGS. 5A-5B illustrate an exemplary detection device, a DMF cartridge, for optical analysis using evanescent-wave-mediated Fourier-Transform Infrared (FTIR) spectroscopy of a fluid (e.g., droplets) containing a target analyte, in accordance with another embodiment of the invention.

As shown in FIGS. 5A-5B, the DMF cartridge 500 and 502 includes a top plate 510 comprising an internal reflectance element (IRE) material 550, a bottom plate 520 including a plurality of PCB substrates 523 and PCB pads 524 operable to perform droplet operations (e.g., droplet manipulation) on a droplet 540 and a gap 530 formed by the separation or spacing between the top plate 510 and the bottom plate 520. Although FIGS. 5A-5B are shown and described using a top plate comprising an internal reflectance element (IRE) material, other embodiments are envisioned herein. For example, in one embodiment, the internal reflectance material can be disposed in the gap between the top plate and the bottom plate. In another embodiment, the top plate can be made from an IRE material, or the IRE material can be embedded in the top plate. In still another embodiment, the bottom plate can be made from an IRE material, or the IRE material can be embedded in the bottom plate.

In general, any known high-refractive index material can be used as the internal reflectance element (IRE) herein. For example, the IRE material can comprise a high-refractive index material 500, such as one selected from Germanium (Ge), Zinc selenide (ZnSe), silicon, amorphous material transmitting infrared radiation (AMTIR) material, diamond or any combination thereof. In some embodiments, the high-refractive index material is selected from a Germanium (Ge) crystal, a Zinc selenide (ZnSe) crystal, a silicon crystal, an amorphous material transmitting infrared radiation (AMTIR) crystal, a diamond crystal or any combination thereof. In some embodiments, the IRE material comprises a surface that can be functionalized with a receptor capable of binding the analyte of interest. For example, the receptor can be directly bound to the crystal surface through hydrogen bonds, hydrophobic interactions or through van der Waals interactions, or the receptor is directly bound to a coating applied to the surface. In another embodiment, the IRE surface can comprise a receptor-functionalized crystalline surface, for example, by using one or more linker molecules to bind the receptor to the IRE surface. For example, the IRE material can comprise a surface and the can be functionalized by bindings the receptor to the IRE surface using one or more linkers, wherein the one or more linkers include common functional groups used for protein-peptide or aptamer immobilization, including one or more amine (NH2) groups, sulfhydryl (SH) groups, aldehyde (CHO) groups, carboxylate (COO—) groups, or a hydroxyl (OH) groups, or any combination thereof. In another embodiment, the receptor can be bound to the IRE surface using cross-linking chemistries such as an NHS ester linkage, a maleimide linkage, a hydrazide linkage, EDC coupling, or a biotin-streptavidin linkage. As an analyte or ligand 562 binds to the receptor-functionalized surface 560, FTIR can be used at the crystalline surface to quantify the analyte of interest. Although FTIR is exemplified in accordance with this embodiment, it would be well understood in the art that other means for performing evanescent-wave-mediated spectroscopy can be used in the practice of the present invention. For example, evanescent-wave-mediated ultraviolet-visible (UV-Vis) spectroscopy, evanescent-wave-mediated Fourier-Transform Infrared (FTIR) spectroscopy, evanescent-wave-mediated Raman spectroscopy, evanescent-wave-mediated Circular Dichroism (CD) spectroscopy, evanescent-wave-mediated Near-InfraRed (NIR) spectroscopy, evanescent-wave-mediated Microfluidic Modulation spectroscopy (MMS) and evanescent-wave-mediated terahertz spectroscopy can be used. In some embodiments, the DMF cartridge and method described herein can be used for both surface plasmon resonance (SPR) and for Fourier-Transform Infrared (FTIR) spectroscopy.

In some embodiments, the internal reflectance element (IRE) material comprises a porous material to maximize the surface area available for ligand capture. In general, any known porous material with a suitable refractive index can be used as the IRE material. For example, in some embodiments, the porous material comprises porous silicon photonic crystals or porous tantalum oxide photonic crystals.

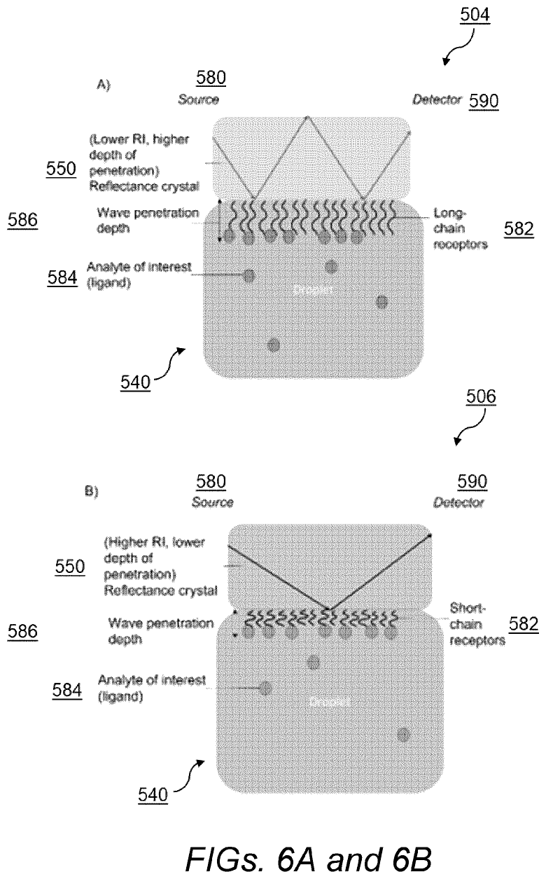

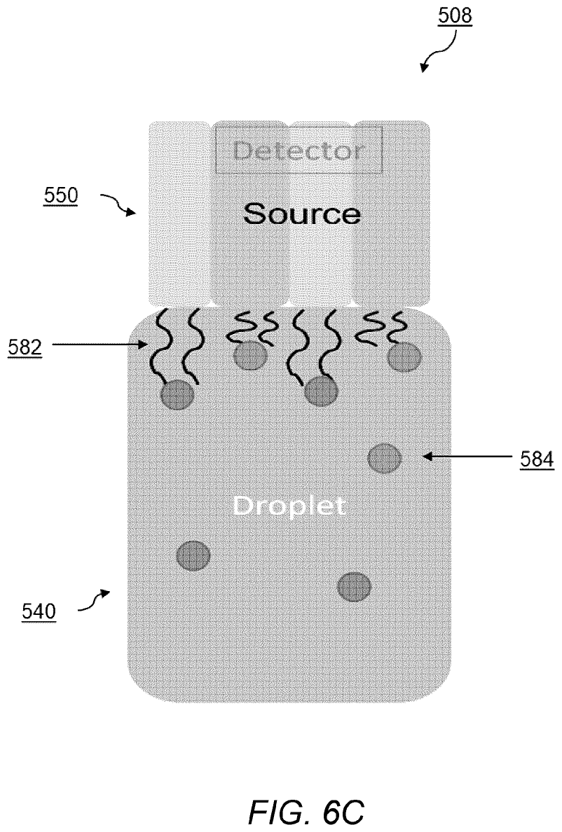

Different IREs produce evanescent waves that penetrate the sample at different depths (depending on their refractive indices), allowing one to probe ligand molecules bound at different distances from the surface of the IRE. For instance, disclosed in FIGS. 6A-6C are receptor molecules of different lengths (which can be used for different applications). Examples of “long-chain” receptors might include antibodies functionalized to the active surface of the IRE via biotin-streptavidin linkages, or other types of linker molecules, or peptide-functionalized hydrogels. In contrast “short-chain” receptors might include functional groups directly bound to the active surface of the crystal, or even non-functionalized crystals (which simply adsorb biomolecules by means of hydrophobic or ionic interactions). In all cases, by changing the IRE we can tune the depth of penetration of the wave into the sample to match the length and size of these receptors/ligands, while also minimizing background from the aqueous bulk.

FIG. 6A-6D illustrate exemplary detection devices, including DMF cartridges for optical analysis using evanescent-wave-mediated Fourier-Transform Infrared (FTIR) spectroscopy of a fluid (e.g., droplets) containing a target analyte, in accordance with another embodiment of the invention. As shown in FIGS. 6A-6D, the DMF cartridges include a top plate comprising one or more internal reflectance element (IRE) materials, wherein each embodiment shown in FIGS. 6A-D are exemplified with a different IRE material. Electromagnetic radiation can be directed from the electromagnetic radiation light source 580 through the internal reflectance element (IRE) material, thereby creating an evanescent wave which penetrates the sample droplet, and subsequently the signal detected at a detector or sensor 590.

In FIG. 6A, the top plate includes a low refractive index IRE surface 550 (such as Zinc selenide (ZnSe) or diamond) providing for a higher depth of penetration 586 of the evanescent wave. In this embodiment, a long chain receptor 582 can be used in combination with the lower refractive index 550 material allowing the DMF cartridge to be tuned for to tune the DMF cartridge for particular analytes of interest 584 (e.g., larger analytes) that can be detected with a higher wave depth penetration 586. In FIG. 6B the top plate includes a higher refractive index IRE surface 550 material (such as Germanium (Ge) or silicon) providing for a lower depth of penetration 586 of the evanescent wave. In this embodiment, a short chain receptor 582 can be used in combination with the higher refractive index material 550 allowing the DMF cartridge to be tuned for to tune the DMF cartridge for particular analytes of interest 584 (e.g., larger analytes). In one embodiment, as shown in FIG. 6C, the IRE material may comprise both a low refractive index IRE material and a higher refractive index IRE material, thereby allowing the DMF cartridge to be used for simultaneous detection (or tuned for detection) of multiple target analytes. In another embodiment, as shown in FIG. 6D, the top plate includes a porous IRE material. The use of a porous IRE material maximizes the surface area available for ligand capture. In the event that multiple samples need to be analyzed at this site, the device can be designed such that the length of the reflecting element (L-crystal) is less than the length of the droplet (L-drop) so that the drop remains in contact with the conductive top plate, and can be actuated away from the reflectance element. As one of skill in the art would appreciate, there is a risk of the sample droplet “pinning” to the rough surface. Therefore, the length of the porous material (L-crystal) can be made shorter than the length of the droplet (or the pad on the bottom plate) (L-drop), to allow the droplet to be actuated away from the crystal using DMF. Any residual liquid remaining in the pores can be removed by passing a washing buffer (or regeneration buffer) over the crystal surface to remove the ligand, before bringing a fresh sample to be analyzed at the crystal.

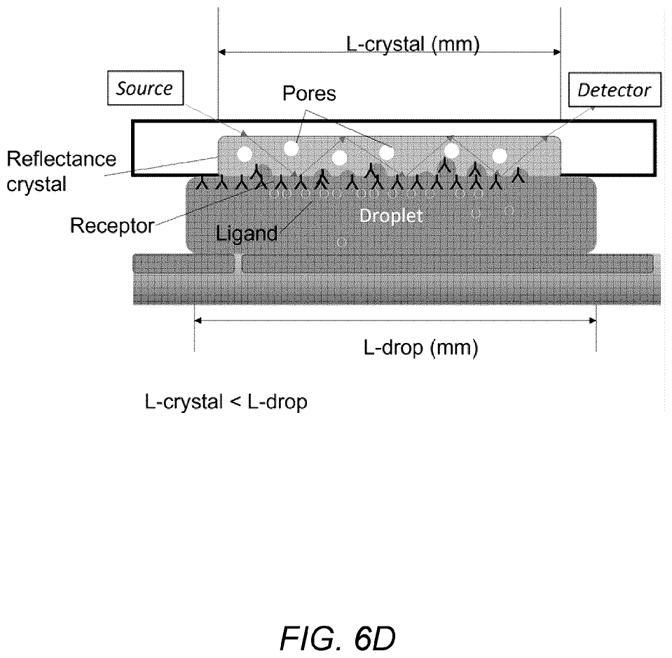

FIG. 7 illustrates a method for optical analysis using Fourier-Transform Infrared (FTIR) spectroscopy of an analyte of interest in a droplet, in accordance with one aspect of the invention. As shown in FIG. 7, the method utilizes a DMF cartridge that has a top plate including an embedded internal reflectance element (IRE) material.

At step 702, a DMF cartridge is provided. In accordance with this aspect of the invention, providing a DMF cartridge, the DMF cartridge comprising: (i) a bottom plate and a top plate, wherein the bottom plate and the top plate are separated to form a gap; (ii) the top plate and/or the bottom plate comprises an embedded internal reflectance element (IRE) material; and (iii) a sample droplet positioned in the gap, wherein the droplet includes an analyte of interest. In general, any DMF cartridge disclosed herein can be used in the practice of this method. In particular, any DMF cartridge comprising a transparent bottom plate and or a transparent bottom plate can be used, such as those disclosed herein in conjunction with FIGS. 5A through 6C.

At step 704, an electromagnetic radiation light source arranged to transmit electromagnetic radiation through the internal reflectance element (IRE) material embedded in the top plate is provided. In accordance with this aspect of the invention, any known evanescent-wave-mediated spectroscopy can be used. For example, the evanescent-wave-mediated spectroscopy can be selected from evanescent-wave-mediated ultraviolet-visible (UV-Vis) spectroscopy, evanescent-wave-mediated Fourier-Transform Infrared (FTIR) spectroscopy, evanescent-wave-mediated Raman spectroscopy, evanescent-wave-mediated Circular Dichroism (CD) spectroscopy, evanescent-wave-mediated Near-InfraRed (NIR) spectroscopy, evanescent-wave-mediated Microfluidic Modulation spectroscopy (MMS) and evanescent-wave-mediated terahertz spectroscopy. In another embodiment, the evanescent-wave-mediated spectroscopy comprises evanescent-wave-mediated Fourier-Transform Infrared (FTIR) spectroscopy.

At step 706, a sensor is provided, wherein the sensor is operable to detect electromagnetic radiation transmitted through the droplet and/or emitted by the droplet.

At step 708, electromagnetic radiation is directed from the electromagnetic radiation light source through the internal reflectance element (IRE) material, thereby creating an evanescent wave which penetrates the sample droplet.

At step 710, electromagnetic radiation and/or the evanescent wave are detected at the sensor.

At step 712, a processor is used to analyze an analyte of interest in the droplet.

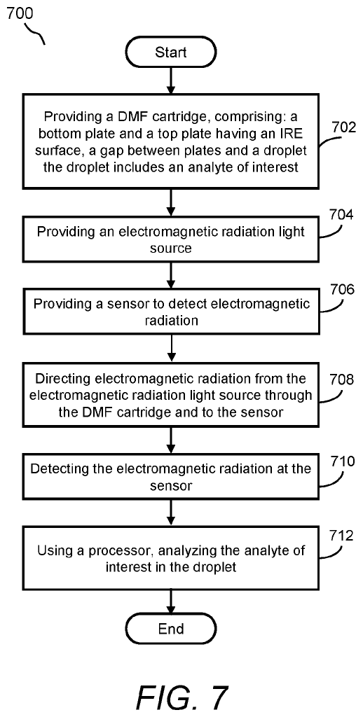

FIGS. 8A-8B illustrate hollow core optical fiber arrangements useful for the practice of one embodiment of the present invention. As shown in FIGS. 8A-8B, the hollow core optical fiber arrangements 800, 802 include a hollow core fiber 810, a sample droplet actuator 820, a fluidic pump 830, an IR light source 840 and a sensor or detector 850. The use of a long hollow channel (e.g., a 10 μm diameter core) allows for a long IR path when analyzing a small fluid volume (e.g., 1 nL). Furthermore, as one of skill in the art would readily understand, the length of the core fiber can be adjusted as needed, for a given application, to increase or decrease the IR path by increasing or decreasing the length of the fiber itself. In accordance with this embodiment, the hollow channel of the hollow core fiber 810 can include an embedded internal reference element (IRE). As described above, in conjunction with FIGS. 5A through 7, the IRE can be used for optical analysis using evanescent-wave-mediated Fourier-Transform Infrared (FTIR) spectroscopy of a fluid (e.g., droplets) containing a target analyte. The hollow core optical fiber arrangement 800, 802 may also optionally have one or more couplers 860 operable to control the IR path length.

Although FIGS. 8A-8B are shown and described with a hollow channel comprising an embedded internal reflectance element (IRE) material, other embodiments are envisioned herein. For example, on one embodiment, the hollow channel can be made from an IRE material. In another embodiment, the internal reflectance element (IRE) material comprises a porous material to maximize the surface area available for ligand capture. In general, any known porous material with a suitable refractive index can be used as the IRE material for, or embedded to, the hollow channel. For example, in some embodiments, the porous material comprises porous silicon photonic crystals or porous tantalum oxide photonic crystals.

In general, any known high-refractive index material can be used as the internal reflectance element (IRE) herein. For example, the IRE material can comprise a refractive index material, such as one selected from Germanium (Ge), Zinc selenide (ZnSe), silicon, amorphous material transmitting infrared radiation (AMTIR) material, diamond or any combination thereof. In some embodiments, the refractive index material is selected from a Germanium (Ge) crystal, a Zinc selenide (ZnSe) crystal, a silicon crystal, an amorphous material transmitting infrared radiation (AMTIR) crystal, a diamond crystal or any combination thereof. In some embodiments, the IRE material comprises a surface that can be functionalized with a receptor capable of binding the analyte of interest. For example, the receptor can be directly bound to the crystal surface through hydrogen bonds, hydrophobic interactions or through van der Waals interactions, or the receptor is directly bound to a coating applied to the surface. In another embodiment, the IRE surface can comprise a receptor-functionalized crystalline surface, for example, by using a linker molecule (e.g., a biotin-streptavidin linkage) to couple the receptor to the IRE surface. As an analyte or ligand binds to the receptor-functionalized surface, FTIR can be used at the crystalline surface to quantify the analyte of interest. Although FTIR is exemplified in accordance with this embodiment, it would be well understood in the art that other means for performing evanescent-wave-mediated spectroscopy can be used in the practice of the present invention. For example, evanescent-wave-mediated ultraviolet-visible (UV-Vis) spectroscopy, evanescent-wave-mediated Fourier-Transform Infrared (FTIR) spectroscopy, evanescent-wave-mediated Raman spectroscopy, evanescent-wave-mediated Circular Dichroism (CD) spectroscopy, evanescent-wave-mediated Near-InfraRed (NIR) spectroscopy, evanescent-wave-mediated Microfluidic Modulation spectroscopy (MMS) and evanescent-wave-mediated terahertz spectroscopy can be used. In some embodiments, the DMF system and method described herein can be used for both surface plasmon resonance (SPR) and for Fourier-Transform Infrared (FTIR) spectroscopy.

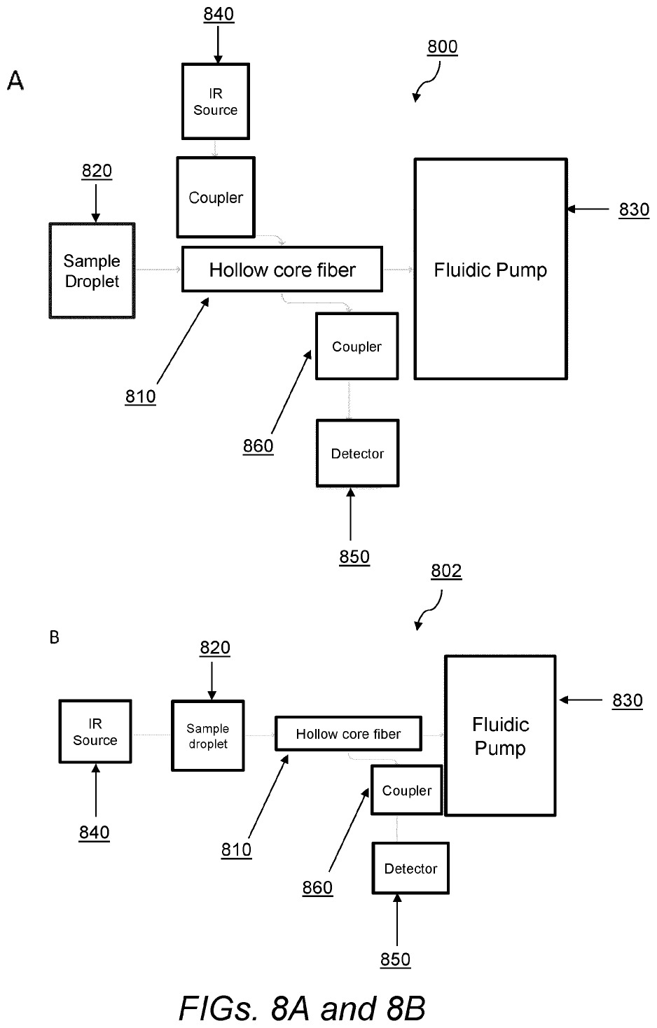

FIG. 9A-9C illustrate exemplary operations of the systems of FIGS. 8A-8B, with optional pump, for fluid transport through the hollow core fiber. As shown, in FIGS. 9A-9C, in accordance with the practice of one embodiment of this invention, the use of an input optical fiber coupler and an output optical fiber coupler adjacent to the hollow core fiber allow the IR path to proceed from the input optical fiber coupler, through the fiber core, and subsequently to the output optical fiber coupler. Also, as shown in FIGS. 9A-9C, optionally using the pump, fluid is pulled through the hollow core fiber for analysis. In one embodiment, the core is pre-seeded with water to prevent oil ingress into it and the pump is used to displace the water with an analyte containing fluid. In another embodiment, the core is pre-seeded with oil. The substantially hydrophilic core will assist the replacement of oil with water. Optionally, in an oil-based device, the hollow fiber automatically seeds with oil when doing cartridge setup. In accordance with this embodiment, the sample displaces the oil to fill the (hydrophobic) fiber by capillary action, and thus, this operation can be completed without the use of a pump (i.e., pump-free). As one of skill in the art would readily appreciate, this operation can be completed through manipulation of the surface properties of the fiber allowing it to pull aqueous fluid into the hollow channel and displace the oil. In still another embodiment, the DMF device is in air (no oil medium). The core is left empty. In this embodiment, the pump is not needed as capillary force will allow the fiber to fill fully.

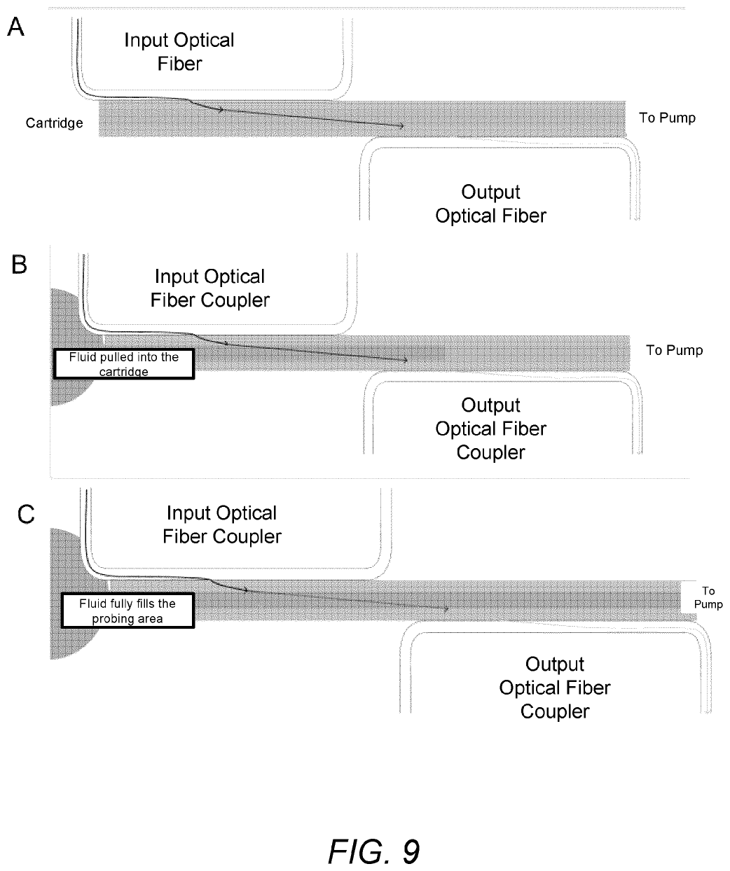

FIG. 10 illustrates Microfluidic modulation spectroscopy, a technique whereby a sample and a reference solvent stream are rapidly modulated through a microfluidic cell in a DMF device, in accordance with one aspect of the present invention. Microfluidic modulation spectroscopy is a technique where a sample and a reference solvent stream are rapidly modulated through a microfluidic cell that is being probed by a laser. Modulation can be used to obtain a differential signal that minimizes aqueous background and drift, and is useful for low-abundance analytes. Prior work has involved the use of this technique on channel architectures, and has been demonstrated for the analysis of the secondary structure of proteins and protein quantitation. This functionality can be extended to other vibrational frequencies to analyze a variety of functional groups, and can be executed on a DMF system, by rapidly exchanging reference buffer droplets with sample droplets over a fiber-sensor surface, or over a dedicated optical analysis electrode. The latter can be done by sending light along the plane of the cartridge or through the cartridge (top to bottom)—this last embodiment will require an IR-transparent cartridge.

As shown in FIG. 10, the DMF cartridge includes a bottom plate 1010 and a top plate 1020 spaced apart to form a gap between the plates. The bottom plate includes a plurality of electrodes 1030 operable to perform droplet operations (e.g., droplet manipulation). In accordance with this embodiment, Microfluidic modulation spectroscopy reference buffer droplets 1070 and sample analyte droplets 1060 alternate over a fiber-sensor surface, or over a dedicated optical analysis electrode and the sample analyte droplets are interrogated using a laser 1040 and detector or sensor 1050 spaced on opposite sides of the DMF cartridge.

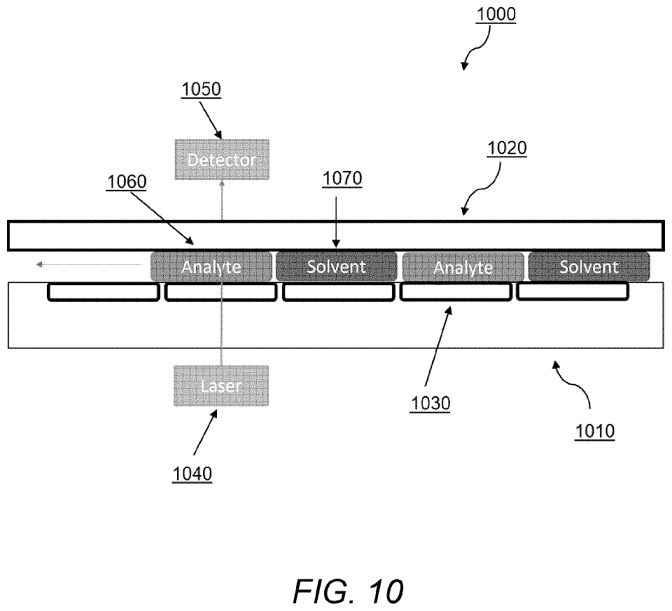

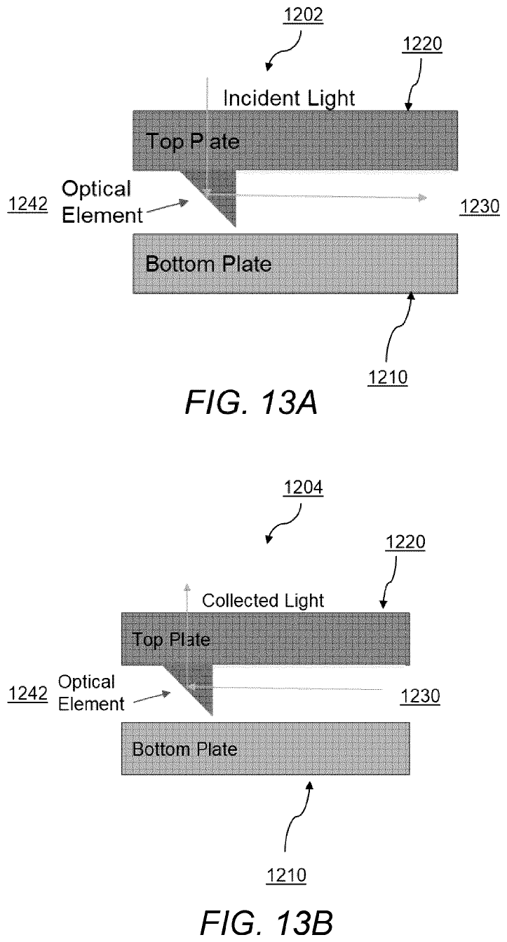

FIG. 11 illustrates a method for optical analysis using electromagnetic spectroscopy of an analyte of interest in a droplet, in accordance with one aspect of the invention. As shown in FIG. 11, the method utilizes a DMF cartridge comprising a bottom plate, a top plate and a gap between the bottom and top plate. The gap further comprises an optical element operable to refract a light path used for interrogation or analysis of a fluid (e.g., a droplet) contained therein, and thereby elongate or increase the light path through the fluid. Although this aspect of the invention is described and exemplified in conjunction with FIGS. 11-16, one or more optical elements can be used. For example, in one embodiment, as shown in conjunction with FIG. 14 a series (e.g., three prisms) optical elements can be used. In other embodiments, from 2 to 10, from 2 to 8, or from 2 to 5 optical elements can be used.

As shown in FIG. 11, a method 1100 is provided for analyzing an analyte of interest in a droplet. At step 1102, providing a DMF cartridge, the DMF cartridge comprising: (i) a bottom plate and a top plate, wherein the bottom plate and the top plate are separated to form a gap, and wherein the gap includes an optical element operable to refract light from a light source; and (ii) a droplet positioned in the gap, wherein the droplet includes an analyte of interest. In some embodiments, the optical element comprises an optically active material, and wherein the optically active material is deposited or doped onto the top plate. In some embodiments, the optical element comprises a prism, and wherein the prism is operable to refract light from a perpendicular light path to a planar light path. Alternatively, in some embodiments, the optical element comprises a prism, and wherein the prism is operable to refract light from a horizontal light path to a perpendicular light path. In other embodiments, the prism comprises a mirrored prism or a dichroic prism. In one embodiment, the optical element comprises a beamsplitter operable to split the light in the gap into two light beams. In one embodiment, the optical element comprises a series of two or more prisms. For example, the series of two or more prisms comprises two or more dichroic prisms with wavelength dependent properties. In some embodiments, the optical element comprises a reflective curved surface, and wherein the reflective curved surface operates to focus the light to a single point within the gap. In one embodiment, the single point within the gap comprises an aperture, and the aperture operates to focus the light onto the sensor. In still another embodiment, a hybrid DMF-channel-microfluidic device can be used, wherein the channel operates to wick the analyte fluid to a well for subsequent analysis by a light source. In accordance with this embodiment, the use of a well to collect a greater volume of analyte fluid for analysis operates to elongate or increase the length of the light path through the analyte fluid and thereby improving measurement accuracy. In still yet another embodiment, the DMF cartridge comprises a bottom plate having one or more electrodes or pads, a top plate spaced apart from the top plate forming a gap between the plates, and further comprises one or more trenches wherein the trenches comprise a void in the bottom plate (e.g., due to absence of an electrode or pad). In accordance with this embodiment, the void in the bottom plate creates a larger gap (e.g., by creating a greater distance in the gap between the bottom plate and the top plate). Droplets naturally migrate and can be held in the void for analysis by a light source. The increased volume (due to the increased gap distance) elongates the light path for this analysis step.

At step 1104, providing a light source arranged to transmit light through the droplet and a sensor arranged to detect light from the droplet, thereby creating a light path through the droplet, and wherein the optical element in the gap elongates the light path through the droplet. In some embodiments, the light source and sensor are arranged horizontally with the bottom plate and top plate and the light source is operable to transmit light horizontally through the droplet to the sensor. In other embodiments, the light source can be arranged horizontally and the sensor perpendicular. In accordance with this embodiment, light can be transmitted from the light source horizontally (i.e., a horizontal or planar light path) through the gap (and optionally through the droplet) between the top and bottom plate, be refracted (e.g., using the optical element described herein) and redirected perpendicular to the top and bottom plates, and subsequently detected by the sensor. In yet another embodiment, as would be readily apparent to one of skill in the art, the light source can be arranged perpendicular to the top and bottom plates and the sensor horizontally to the top and bottom plates. In accordance with this embodiment, light can be transmitted from the light source vertically (i.e., a perpendicular light path), be refracted (e.g., using the optical element described herein) and redirected horizontally through the gap (and optionally through the droplet), and subsequently detected by the sensor.

At step 1106, directing light from the light source through the droplet and to the sensor and at step 1108 detecting the light at the sensor.

At step 1110, using a processor, analyzing the analyte of interest in the droplet.

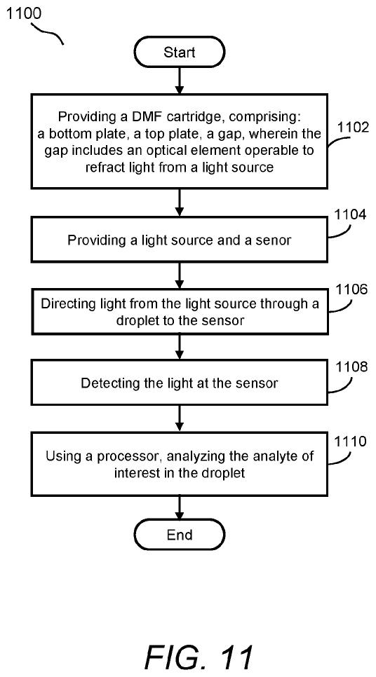

FIG. 12 illustrates an exemplary absorbance detection device, a DMF cartridge, for optical analysis of an analyte of interest in a droplet using electromagnetic spectroscopy, as described in FIG. 11. As shown in FIG. 12, the exemplary DMF cartridge 1200 comprises a bottom plate 1210, a top plate 1220 spaced apart from the bottom plate 1210 forming a space or gap 1230 between the plates, and an optical element 1240 in the gap operable to refract light from a light source, and thereby elongate the light path. Furthermore, as shown in FIG. 12, the optically element 1240 comprises an optically active material 1250 that is deposited or doped onto the top plate.

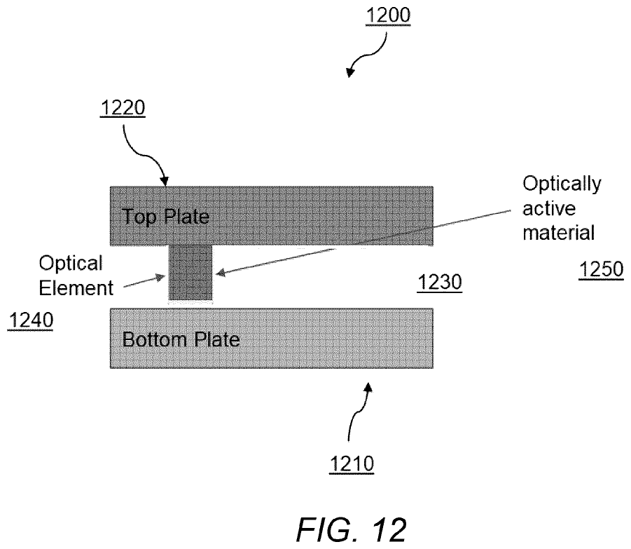

FIG. 13A-B illustrate exemplary detection devices, including DMF cartridges for optical analysis of an analyte of interest in a droplet using electromagnetic spectroscopy, as described in FIG. 11. As shown in FIG. 13A, the exemplary DMF cartridge 1202 comprises a bottom plate 1210, a top plate 1220 spaced apart from the bottom plate 1210 forming a space or gap 1230 between the plates, and an optical element 1242 in the gap operable to refract light from a light source, and thereby elongate the light path. Furthermore, as shown in FIG. 13A, the optical element comprises a prism, and wherein the prism is operable to refract light from a perpendicular light path to a planar or horizontal light path. Whereas, as shown in FIG. 13B, the exemplary DMF cartridge 1204 comprises a bottom plate 1210, a top plate 1220 spaced apart from the bottom plate 1210 forming a space or gap 1230 between the plates, and an optical element 1242 in the gap operable to refract light from a light source, and thereby elongate the light path. In FIG. 13A, the optical element comprises a prism, wherein the prism is operable to refract light from a planar or horizontal light path to a perpendicular or vertical light path. The prism can be a mirrored prism or a dichroic prism.

FIG. 14 illustrates an exemplary detection device, a DMF cartridge, for optical analysis of an analyte of interest in a droplet using electromagnetic spectroscopy, as described in FIG. 11. As shown in FIG. 14, the exemplary DMF cartridge 1204 comprises a bottom plate 1210, a top plate 1220 spaced apart from the bottom plate 1210 forming a space or gap 1230 between the plates, and an optical element 1244 in the gap operable to refract light from a light source, and thereby elongate the light path. Furthermore, as shown in FIG. 12, the optical element comprises a series of three prisms 1244. The series of three prisms comprises three different dichroic prisms with wavelength dependent properties allowing for separate analysis of different wavelengths of electromagnetic radiation, as shown. Although, as noted above, other arrangements of optical elements can be used in the practice of this aspect of the invention. For example, from 2 to 10, from 2 to 8, or from 2 to 5 optical elements can be used.

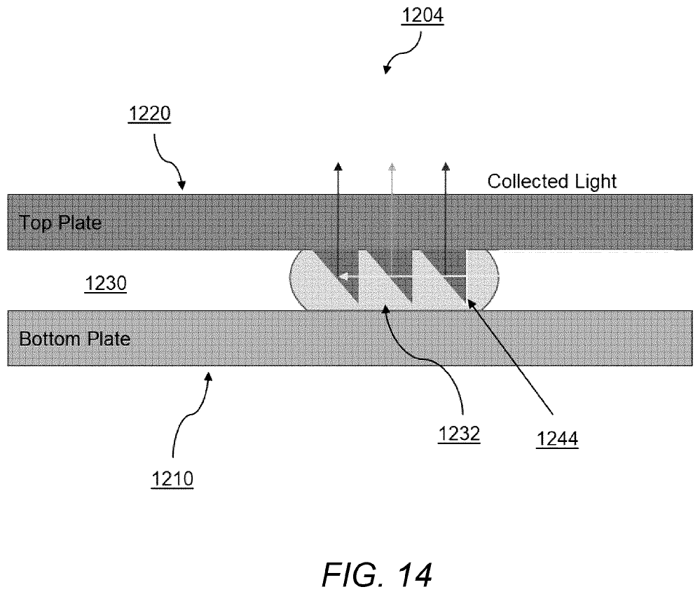

FIG. 15 illustrates an exemplary detection device, a DMF cartridge, for optical analysis of an analyte of interest in a droplet using electromagnetic spectroscopy, as described in FIG. 11. As shown in FIG. 15, the exemplary DMF cartridge 1206 comprises a bottom plate 1210, a top plate 1220 spaced apart from the bottom plate 1210 forming a space or gap 1230 between the plates, and an optical element 1246 in the gap operable to refract light from a light source, and thereby elongate the light path. Furthermore, as shown in FIG. 15, the optical element comprises a reflective curved surface 1246, wherein the reflective curved surface operates to focus the light to a single point within the gap.

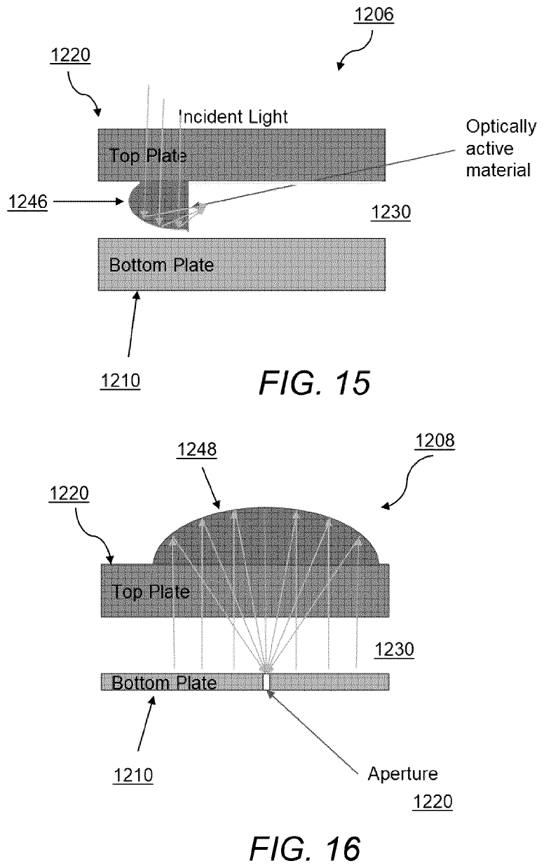

FIG. 16 illustrates an exemplary detection device, a DMF cartridge, for optical analysis of an analyte of interest in a droplet using electromagnetic spectroscopy, as described in FIG. 11. As shown in FIG. 16, the exemplary DMF cartridge 1208 comprises a bottom plate 1210, a top plate 1220 spaced apart from the bottom plate 1210 forming a space or gap 1230 between the plates, and an optical element 1248 in the gap operable to refract light from a light source, and thereby elongate the light path. Furthermore, as shown in FIG. 16, the optical element comprises a reflective curved surface 1248, wherein the reflective curved surface operates to focus the light to a single point, through an aperture in the bottom plate 1210, and onto the sensor or detector.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the invention shall also cover any such alternatives, modifications, variations or equivalents. It is intended that the following claims define the scope of the disclosure and that methods and structures within the scope of these claims and their equivalents be covered thereby.

What is claimed is:

1. A digital microfluidic (DMF) cartridge, the cartridge comprising:
a bottom plate, the bottom plate comprising a bottom plate substrate and a plurality of electrodes operable to perform droplet operations;
a top plate, the top plate comprising a top plate substrate;
wherein the top plate and the bottom plate are separated to form a gap; and
wherein the bottom plate substrate and/or the top plate substrate comprise a material that is transparent to one or more wavelengths of electromagnetic radiation or wherein the bottom plate substrate and/or top plate substrate comprise a through hole that is transparent to one or more wavelengths of electromagnetic radiation.

2. The DMF cartridge of claim 1, wherein the bottom plate substrate and/or top plate substrate are made from a material that is transparent to one or more wavelengths of electromagnetic radiation, and wherein the bottom plate, the gap and the top plate comprise a transparent pathway through which one or more wavelengths of electromagnetic radiation can be transmitted therethrough.

3. The DMF cartridge of claim 1, wherein the bottom plate substrate is made from a material that is transparent to one or more wavelengths of electromagnetic radiation selected from x-ray, ultraviolet light, visible light, infrared light, microwave and any combination thereof.

4. The DMF cartridge of claim 1, wherein the top plate substrate is made from a material that is transparent to one or more wavelengths of electromagnetic radiation selected from x-ray, ultraviolet light, visible light, infrared light, microwave and any combination thereof.

5. The DMF cartridge of claim 1, wherein the top plate substrate and the bottom plate substrate are both transparent to one or more wavelengths of electromagnetic radiation selected from x-ray, ultraviolet light, visible light, infrared light, microwave and any combination thereof.

6. The DMF cartridge of claim 2, wherein the material that is transparent to one or more wavelengths is selected from quartz, cyclo olefin polymer (COP), Cyclic olefin copolymer (COC), a ceramic, a multi-layer flexible PCB transmissible to visible light and any combination thereof.

7. The DMF cartridge of claim 1, wherein the bottom plate substrate is coated with a transparent conductive material.

8. The DMF cartridge of claim 1, wherein the top plate substrate is coated with a transparent conductive material.

9. The DMF cartridge of claim 27, wherein the transparent conductive material is indium tin oxide (ITO).

10. The DMF cartridge of claim 1, wherein the bottom plate substrate further comprises a material capable of filtering out one or more wavelengths of electromagnetic radiation.

11. The DMF cartridge of claim 1, wherein the top plate substrate further comprises a material capable of filtering out one or more wavelengths of electromagnetic radiation.

12. The DMF cartridge of claim 1, wherein the plurality of electrodes comprises an actuation grid.

13. The DMF cartridge of claim 1, wherein the DMF cartridge has the same dimensions as a standard well plate.

14. The DMF cartridge of claim 1, wherein the transparency of the bottom plate substrate and/or top plate substrate coincides with the wells of a standard well plate.

15. A method for analyzing an analyte of interest in a droplet using electromagnetic spectroscopy, the method comprising:
providing a digital microfluidic (DMF) cartridge;
providing an electromagnetic radiation light source arranged to transmit electromagnetic radiation to a droplet disposed in the DMF cartridge;
providing a sensor operable to detect electromagnetic radiation transmitted from the droplet;
directing electromagnetic radiation from the electromagnetic radiation light source to the droplet;
detecting the electromagnetic radiation at the sensor; and
using a processor, analyzing an analyte of interest in the droplet.

16. The method of claim 15, wherein the DMF cartridge comprises:
a bottom plate, the bottom plate comprising a bottom plate substrate and a plurality of electrodes operable to perform droplet operations;
a top plate, the top plate comprising a top plate substrate;
wherein the top plate and the bottom plate are separated to form a gap; and
wherein the bottom plate substrate and/or the top plate substrate comprise a material that is transparent to one or more wavelengths of electromagnetic radiation or wherein the bottom plate substrate and/or top plate substrate comprise a through hole that is transparent to one or more wavelengths of electromagnetic radiation.

17. The method of claim 15, wherein the electromagnetic spectroscopy is selected from ultraviolet-visible (UV-Vis) spectroscopy, Fourier-Transform Infrared (FTIR) spectroscopy, Raman spectroscopy, Circular Dichroism (CD) spectroscopy, Near-InfraRed (NIR) spectroscopy, Microfluidic Modulation spectroscopy (MMS) and terahertz spectroscopy.

18. The method of claim 15, wherein the electromagnetic radiation light source is an optical fiber.

19. The method of claim 18, wherein the sensor is a spectrophotometer.

20. A system for analyzing an analyte in a droplet, the system comprising:
a digital microfluidic (DMF) cartridge, the DMF cartridge comprising a bottom plate and a top plate, wherein the bottom plate and the top plate are separated to form a gap, and wherein the top plate and/or the bottom plate are made of a material that is transparent to one or more wavelengths of electromagnetic radiation; and
an electromagnetic radiation light source capable of emitting electromagnetic radiation, and
a sensor capable of detecting electromagnetic radiation.

21.-86. (canceled)
